(19) United States
(12) Patent Application Publication
(10) Pub. No.: US 2013/0079713 A1
(43) Pub. Date: Mar. 28, 2013

(54) Automatic retractable safety syringe

(71) Applicant: Chih Feng Ho, Taipei (TW)

(72) Inventor: Chih Feng Ho, Taipei (TW)

(73) Assignee: Oxti Corporation

(21) Appl. No.: 13/200,664

(22) Filed: Sep. 28, 2011

(51) Int. Cl. A61M 5/32; A61M 5/50

(52) U.S. Cl. 604/110; 604/198

(57) ABSTRACT

A syringe includes a barrel having a mouth on one end portion, a needle having a nipple engaged in the mouth of the barrel, a plunger slidably engaged into the barrel, a piston releasably attached to the plunger and having a protrusion for engaging with a needle mounting, and a follower slidably engaged in the plunger and having a catch device for securing the piston to the follower, and a spring biasing member engaged with the follower for biasing and retracting the follower and the piston and the needle mounting and the needle into the barrel when the latch member of the latch device is disengaged from the needle mounting and for preventing the needle from being exposed after use.

### BACKGROUND OF THE INVENTION

1. Field of the Invention

The present invention relates to an automatic retractable safety syringe, and more particularly to an automatic retractable safety syringe including a simplified structure that may be easily and quickly manufactured and assembled and that may include a greatly reduced or decreased manufacturing cost and that may be easily actuated or operated by the user.

2. Description of the Prior Art

Typical syringes comprise a piston slidably received or engaged in an outer cylindrical member or barrel, and a piston rod attached or mounted or secured or coupled to the piston for moving the piston along or relative to the cylindrical member or barrel, and for selectively drawing the fluid or medicine into and out of the cylindrical member or barrel.

Due to the recent advent of the AIDS virus, which may be contracted by contaminated hypodermic syringes, there have been several retractable needle hypodermic syringes invented and patented.

For example, U.S. Pat. No. 5,785,687 to Saito, U.S. Pat. No. 6,093,171 to Huang, U.S. Pat. No. 6,196,997 to Saito, and U.S. Pat. No. 6,482,174 to Tiu disclose several of the typical retractable safety syringes each comprising a hollow plunger slidably received or engaged in an outer cylindrical member or barrel, and a piston slidably received or engaged in the plunger for retracting the needle into the plunger after use.

However, in the typical retractable safety syringes, the users have to draw the needle into the plunger after use by themselves, and the needle may not be drawn into the plunger automatically without being operated by the user.

U.S. Pat. No. 7,476,215 to Lam discloses one of the typical automatic retractable safety syringes comprising a hollow plunger slidably received or engaged in an outer cylindrical member or barrel, and a piston slidably received or engaged in the plunger, and a piston vacuum seal required to be attached or mounted or secured to the distal end of the piston, the relative axial movement of the plunger in relation to the piston causes a vacuum to be created within the plunger.

However, the typical automatic retractable safety syringes comprises a number of parts or elements that are required to be precisely assembled. In addition, the piston vacuum seal that is attached or mounted or secured to the distal end of the piston has a good chance to be tilted or inclined relative to the plunger, and the vacuum created within the plunger may have a good chance to become failure.

The present invention has arisen to mitigate and/or obviate the afore-described disadvantages of the conventional automatic retractable safety syringes.

### SUMMARY OF THE INVENTION

The primary objective of the present invention is to provide an automatic retractable safety syringe including a simplified structure that may be easily and quickly manufactured and assembled and that may include a greatly reduced or decreased manufacturing cost and that may be easily actuated or operated by the user.

In accordance with one aspect of the invention, there is provided a syringe comprising a barrel including a chamber formed therein, and including an opening formed in a first end portion thereof, and including a mouth formed in a second end portion thereof, and including a latch device provided in the second end portion thereof, and the latch device including a latch member extended into the mouth, a needle mounting engaged into the barrel and releasably engaged with the latch member of the latch device which is capable or releasing the needle mounting from the barrel for allowing the needle mounting to be released and detached from the barrel and to be engaged into the chamber of the barrel when the latch member of the latch device is disengaged from the needle mounting, the needle mounting including a bore formed therein, a needle including a nipple engaged in the mouth of the barrel and coupled to the needle mounting, a plunger slidably engaged into the opening and the chamber of the barrel, and including a first end portion located out of the barrel, and including a compartment formed therein, and including a second end portion, a piston releasably attached to the second end portion of the plunger and including a protrusion for selectively engaging with the bore of the needle mounting and for selectively securing the piston to the needle mounting, and a follower slidably engaged in the plunger and including a catch device for selectively engaging with the piston and for selectively securing the piston to the follower when the piston is moved toward the follower with the plunger, and a spring biasing member engaged with the follower for biasing and forcing the follower and the piston and the needle mounting and the needle toward the first end portion of the plunger and for biasing and forcing and retracting the needle mounting and the needle into the barrel when the latch member of the latch device is disengaged from the needle mounting.

The plunger includes a guide channel formed therein, and the follower includes a projection slidably engaged with the guide channel of the plunger and for guiding the follower to move relative to the plunger and for limiting the follower to slide relative to the plunger.

The needle mounting includes a retaining member for releasably engaging with the latch member of the latch device which is capable or releasing the needle mounting from the barrel for allowing the needle mounting to be released and detached from the barrel and to be engaged into the chamber of the barrel when the latch member of the latch device is disengaged from the needle mounting.

The latch device includes an inclined surface formed in the latch member for being actuated to disengage the latch member of the latch device from the needle mounting. The barrel includes a gasket disposed therein for selectively engaging with the plunger which selectively forces the gasket to engage with the inclined surface of the latch member.

The barrel includes a space formed therein for receiving and engaging with the latch device. The latch device is resiliently secured to the barrel with a support member. The barrel includes an anchoring member provided in the mouth, and the needle includes an engaging member for releasably engaging with the anchoring member of the barrel.

The piston includes at least one rib extended radially and outwardly therefrom for engaging with the plunger and for releasably attaching to the plunger and for allowing the piston to be selectively disengaged from the plunger.

The piston includes an extension extended outwardly therefrom for selectively engaging with the catch device of the follower and for allowing the follower to be selectively anchored and secured to the piston when the plunger is pulled outwardly relative to the barrel.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is an exploded view of an automatic retractable safety syringe in accordance with the present invention;

FIG. 2 is an enlarged partial perspective view of the automatic retractable safety;

FIG. 3 is a cross sectional view of the automatic retractable safety; and

FIGS. 4, 5, 6, 7, 8, 9 are cross sectional views similar to FIG. 3, illustrating the operation of the automatic retractable safety syringe.

### DETAILED DESCRIPTION OF THE PREFERRED EMBODIMENT

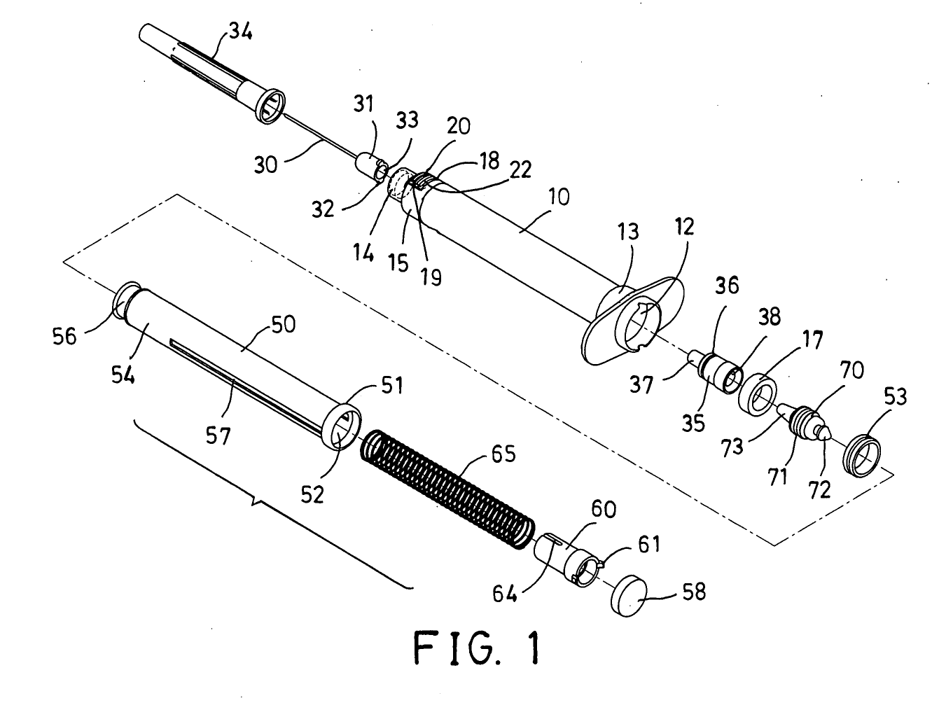

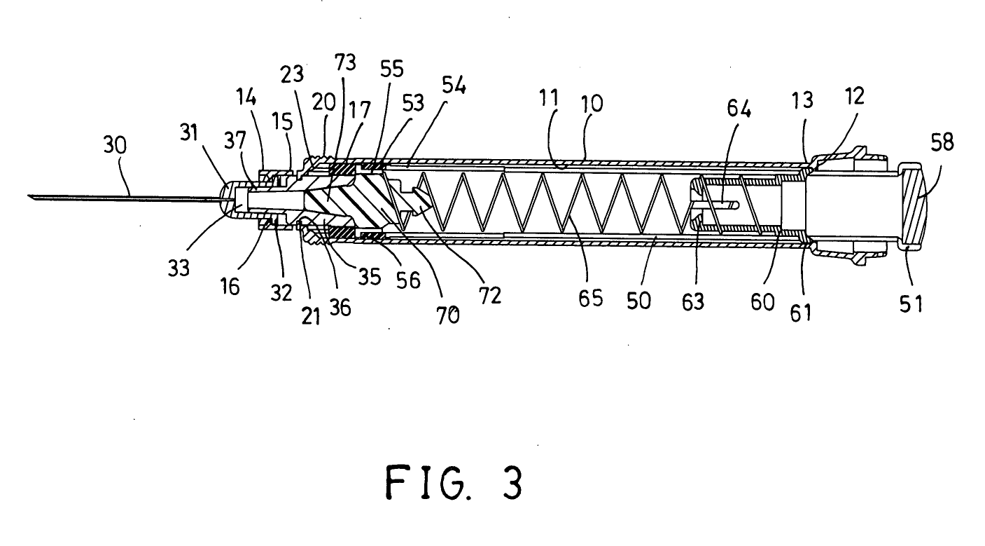

Referring to the drawings, and initially to FIGS. 1-3, an automatic retractable safety syringe in accordance with the present invention comprises a barrel 10 including a longitudinal chamber 11 formed therein, and including an opening 12 formed or provided in the first or one end portion 13 thereof for slidably receiving or engaging with a plunger 50, and including a narrowed mouth 14 formed or provided in the second or other end portion 15 thereof and having an outer diameter smaller than that of the barrel 10, and including an anchoring member 16, such as a catch or latch or projection or threaded member 16 formed or provided or extended or located in the mouth 14, and including a sealing ring or gasket 17 disposed or engaged into the chamber 11 of the barrel 10 and contacted or located close to the mouth 14 of the barrel 10, best shown in FIGS. 3-9.

The barrel 10 further includes one or more (such as two) spring or resilient latch devices 20 formed or provided or extended on or in the second or other end portion 15 thereof, and the latch devices 20 each include an engaging tongue or latch member 21 partially or slightly extended into the mouth 14. For example, the barrel 10 includes one or more (such as two) openings or spaces 18 formed or provided in the second or other end portion 15 thereof for receiving or engaging with the latch devices 20 respectively, in which the latch devices 20 are resiliently secured or coupled to the barrel 10 with a peg or rib or beam or support member 19 for allowing the latch devices 20 to be resiliently attached or mounted or secured or coupled to the barrel 10.

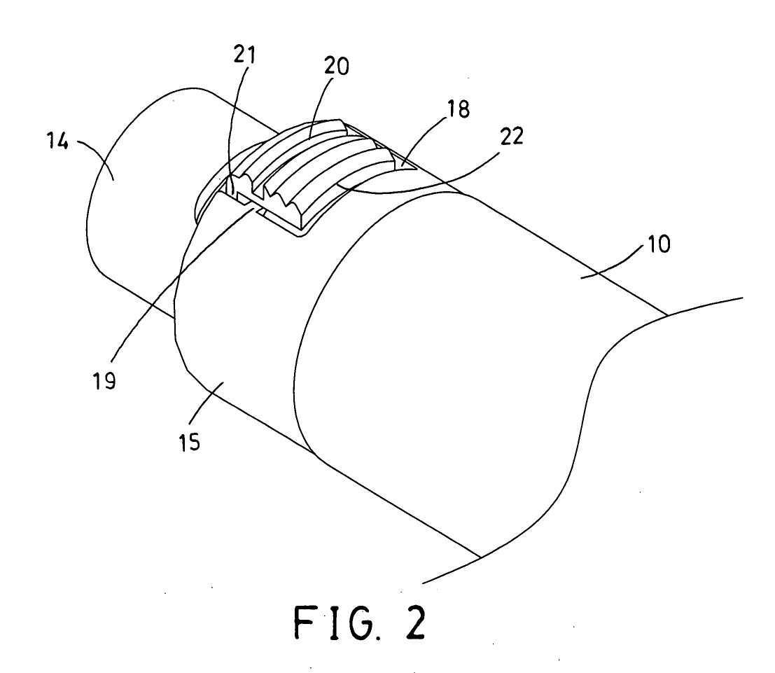

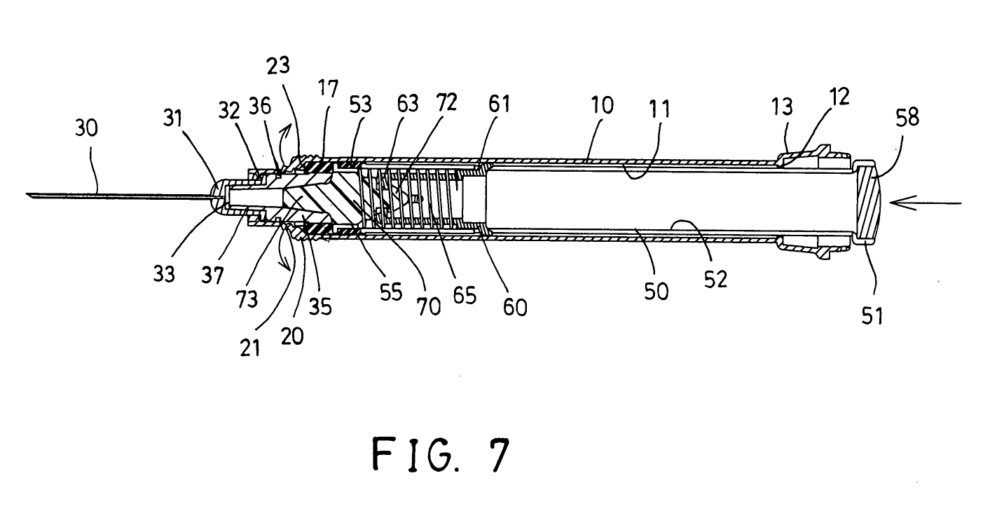

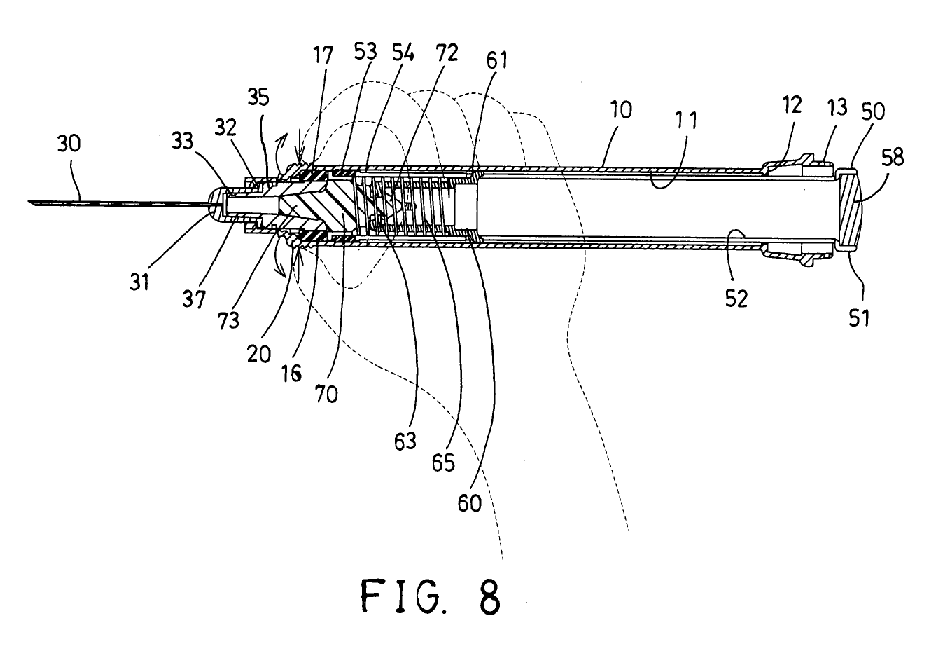

As shown in FIG. 2, it is preferable that the support member 19 is attached or mounted or secured to the middle portion of the latch devices 20 and arranged for allowing the latch member 21 to be moved out of the mouth 14 when an inner end or a hand grip 22 of the latch device 20 is depressed toward or into the barrel 10 or actuated by the user (FIG. 8). As shown in FIG. 3, the latch devices 20 of the barrel 10 each further include a tilted or tapered or inclined surface 23 formed therein, and also arranged for allowing the latch member 21 to be moved out of the mouth 14 when the gasket 17 is forced to move onto and to engage with the inclined surface 23 of the latch device 20 (FIG. 7) and to push or move the latch member 21 of the latch device 20 out of the mouth 14.

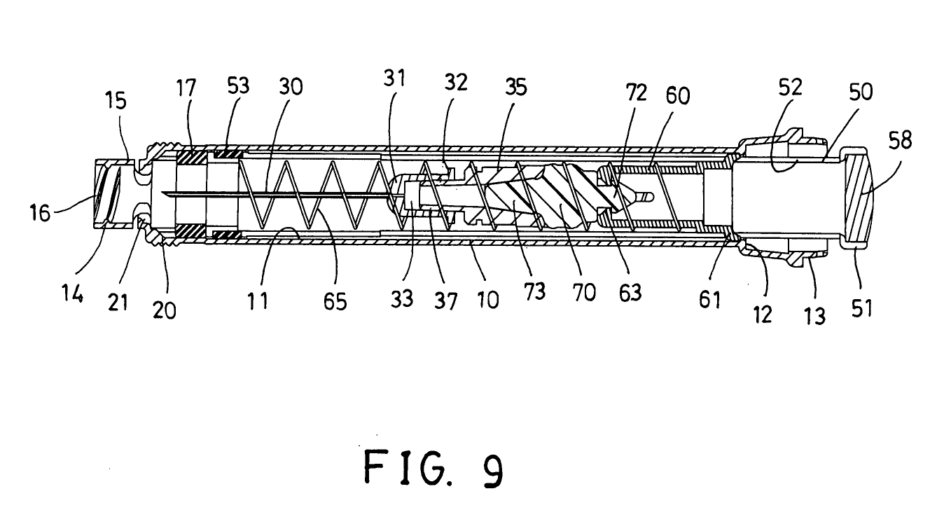

A needle 30 includes a nipple 31 releasably disposed or engaged into the mouth 14 of the barrel 10 and movable or engageable into the chamber 11 of the barrel 10 (FIG. 9), for example, the nipple 31 includes another anchoring member or engaging member 32 formed or provided or extended thereon for selectively and releasably engaging with the anchoring member 16 of the barrel 10 and arranged for allowing the nipple 31 and the needle 30 to be selectively drawn or moved or retracted or pulled into the chamber 11 of the barrel 10. The nipple 31 includes a cavity 33 formed therein, and a protective shield 34 (FIG. 1) may further be provided and attached or mounted or secured or engaged onto or with the needle 30 and the nipple 31 for shielding and protecting the needle 30 before use.

A needle mounting 35 is disposed or engaged into the chamber 11 of the barrel 10, and includes a retaining member 36, such as an outer peripheral slot 36 formed in the outer peripheral portion thereof for selectively and releasably or detachably engaging with the latch members 21 of the latch devices 20; i.e., the latch members 21 of the latch devices 20 may be releasably or detachably engaged with the outer peripheral slot or retaining member 36 of the needle mounting 35 and arranged for allowing the needle mounting 35 to be released or detached from the barrel 10 and to be received or engaged in the chamber 11 of the barrel 10 when the latch members 21 of the latch devices 20 are disengaged from the needle mounting 35. The needle mounting 35 includes a port 37 extended or engaged into the cavity 33 of the nipple 31 and anchored or secured to the nipple 31 with a force-fitted engagement or adhesive materials or welding processes, or the like, and arranged for allowing the needle mounting 35 and the nipple 31 and the needle 30 to be selectively moved into the chamber 11 of the barrel 10 together when the latch members 21 of the latch devices 20 are disengaged from the needle mounting 35. The needle mounting 35 includes a bore 38 formed therein.

The plunger 50 is slidably received or engaged in the chamber 11 of the barrel 10, and includes a first or one end portion 51 extended or located out of the barrel 10, and includes a longitudinal compartment 52 formed therein, and includes a gasket or sealing ring 53 disposed or engaged onto the outer peripheral portion of the second or other end portion 54 of the plunger 50 and contacted or engaged with the barrel 10 for making an air or water or fluid tight seal between the barrel 10 and the plunger 50. For example, the plunger 50 includes an inner peripheral flange 55 formed or extended in the second or other end portion 54 thereof for forming or defining a peripheral recess 56 in the outer peripheral portion thereof for receiving or engaging with the sealing ring 53 and for solidly and stably attaching or mounting or anchoring or retaining the sealing ring 53 to the plunger 50.

The plunger 50 further includes one or more (such as two) grooves or guide channels 57 longitudinally and oppositely formed therein (FIG. 1), and includes a cover or lid 58 attached or mounted or secured or engaged into the one end portion 51 of the plunger 50 for sealing or blocking or enclosing the compartment 52 of the plunger 50. An insert or sliding member or follower 60 is slidably received or engaged in the compartment 52 of the plunger 50, and includes one or more (such as two) keys or catches or projections 61 extended outwardly therefrom for slidably engaging with the guide channels 57 of the plunger 50 and for guiding the follower 60 to slide or move along or relative to the plunger 50, and includes a spring chuck or fetch or grip or grasp or catch device 63 formed or provided therein, and may include one or more slits 64 (FIG. 1) formed therein for increasing the resilience of the catch device 63. The projections 61 of the follower 60 may also be engaged with the first or one end portion 13 of the barrel 10, or the first or one end portion 13 of the barrel 10 may be engaged with the projections 61 of the follower 60 for preventing the follower 60 from moving out through the first or one end portion 13 of the barrel 10.

A spring biasing member 65 may further be provided and disposed or engaged into the compartment 52 of the plunger 50 and engaged with the follower 60, and/or engaged with the inner peripheral flange 55 of the plunger 50 for biasing or forcing or moving the follower 60 toward the one end portion 51 of the plunger 50 or toward the lid 58, or for biasing or forcing or moving the follower 60 away from the second or other end portion 54 of the plunger 50 (FIGS. 3, 9). A piston 70 is releasably or detachably fitted or attached or mounted or secured or anchored or retained in the second or other end portion 54 of the plunger 50 with a force-fitted engagement or adhesive materials or welding processes, or the like, for example, the piston 70 includes one or more peripheral ribs 71 extended radially and outwardly therefrom for engaging with the plunger 50 and for anchoring or securing to the plunger 50 with the force-fitted engagement.

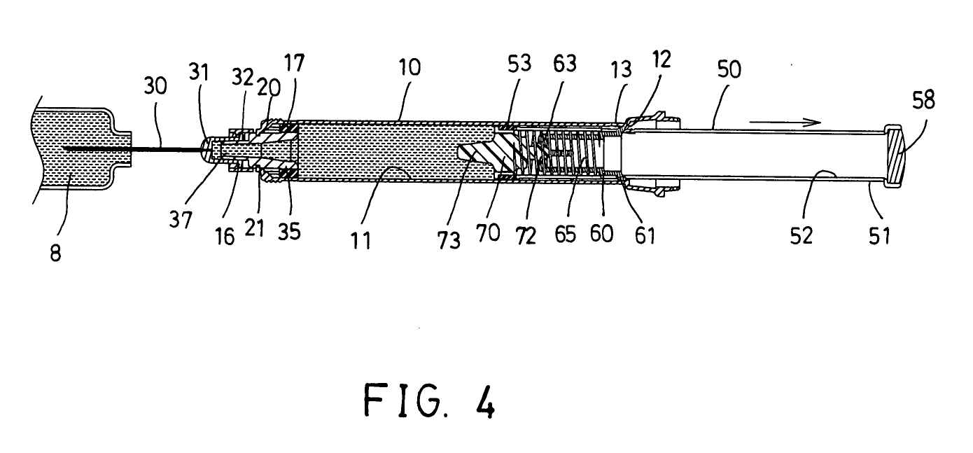

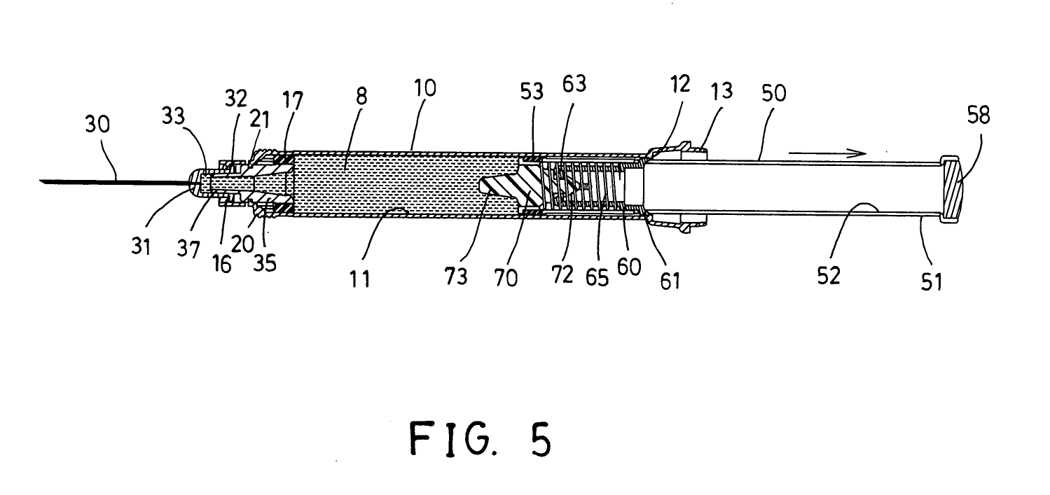

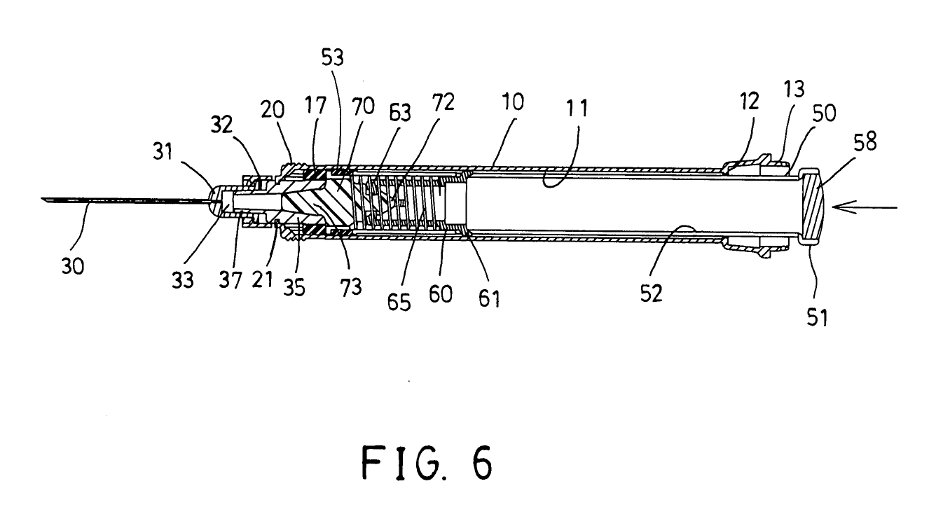

The piston 70 includes an extension 72 extended outwardly therefrom for selectively engaging with the catch device 63 of the follower 60 and arranged for allowing the follower 60 to be selectively anchored or secured to the piston 70 when the plunger 50 is pulled or moved outwardly relative to the barrel 10 (FIGS. 4, 5) or when the piston 70 is moved toward and to engage with the catch device 63 of the follower 60 with the plunger 50, and includes a protrusion 73 for selectively engaging with or into the bore 38 of the needle mounting 35 and for selectively attaching or mounting or securing the piston 70 to the needle mounting 35 (FIGS. 6, 7), and/or for selectively attaching or mounting or securing the needle mounting 35 to the piston 70, and arranged for allowing the needle mounting 35 and the nipple 31 and the needle 30 and the piston 70 and the follower 60 to be selectively moved into the chamber 11 of the barrel 10 together with the spring biasing member 65 when the latch members 21 of the latch devices 20 are disengaged from the needle mounting 35 (FIG. 9).

In operation, as shown in FIG. 4, when the plunger 50 is drawn or moved or pulled out of the barrel 10 or away from the needle 30, the fluid or medicine 8 may be selectively drawn into the chamber 11 of the barrel 10 with the piston 70, at this moment, due to the engagement of the projections 61 of the follower 60 with the guide channels 57 of the plunger 50, the follower 60 may be retained in the first or one end portion 13 of the barrel 10 and will not be moved or pulled out of the barrel 10. As shown in FIG. 5, when the plunger 50 is further drawn or moved or pulled out of the barrel 10 or away from the needle 30, the extension 72 of the piston 70 may be forced to engage with the catch device 63 of the follower 60 for allowing the piston 70 to be selectively anchored or secured or coupled to the follower 60.

The fluid or medicine 8 may then be selectively forced to move or to flow out through the needle 30 for injection purposes by moving or pressing the plunger 50 into the barrel 10 (FIG. 6), and the protrusion 73 of the piston 70 may then be selectively forced to engage with or into the bore 38 of the needle mounting 35 and may then be selectively attached or mounted or secured or anchored or retained to the needle mounting 35. As shown in FIG. 7, after the fluid or medicine injection operation, the plunger 50 may further be pushed or pressed or forced to move toward the nipple 31 and the needle 30 and to contact and to engage with the gasket 17, or to directly engage with the inclined surface 23 of the latch devices 20, and to push or press or force the latch members 21 of the latch devices 20 to move and to be disengaged from the outer peripheral slot or the retaining member 36 of the needle mounting 35, in order to release the needle mounting 35.

The latch members 21 of the latch devices 20 may also be forced to be disengaged from the outer peripheral slot or the retaining member 36 of the needle mounting 35 by depressing the hand grip 22 of the latch device 20 (FIG. 8). When the plunger 50 is further pushed or pressed or forced to move toward the nipple 31 and the needle 30 and to compress or squeeze the gasket 17, the piston 70 may be forced to move or to slide and to be released from the plunger 50, due to a reaction force, or the releasable engagement between the plunger 50 and the piston 70 may be spoiled, and the spring biasing member 65 may bias and force and move and retract the needle mounting 35 and the nipple 31 and the needle 30 and the piston 70 and the follower 60 into the chamber 11 of the barrel 10 (FIG. 9).

Accordingly, the automatic retractable safety syringe in accordance with the present invention includes a simplified structure that may be easily and quickly manufactured and assembled and that may include a greatly reduced or decreased manufacturing cost, and that may be easily actuated or operated by the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A syringe comprising:
a barrel including a chamber formed therein, and including an opening formed in a first end portion thereof, and including a mouth formed in a second end portion thereof, and including a latch device provided in said second end portion thereof, and said latch device including a latch member extended into said mouth,
a needle mounting engaged into said barrel and releasably engaged with said latch member of said latch device which is capable or releasing said needle mounting from said barrel for allowing said needle mounting to be released and detached from said barrel and to be engaged into said chamber of said barrel when said latch member of said latch device is disengaged from said needle mounting, said needle mounting including a bore formed therein,
a needle including a nipple engaged in said mouth of said barrel and coupled to said needle mounting,
a plunger slidably engaged into said opening and said chamber of said barrel, and including a first end portion located out of said barrel, and including a compartment formed therein, and including a second end portion,
a piston releasably attached to said second end portion of said plunger and including a protrusion for selectively engaging with said bore of said needle mounting and for selectively securing said piston to said needle mounting, and
a follower slidably engaged in said plunger and including a catch device for selectively engaging with said piston and for selectively securing said piston to said follower when said piston is moved toward said follower with said plunger, and
a spring biasing member engaged with said follower for biasing and forcing said follower and said piston and said needle mounting and said needle toward said first end portion of said plunger and for biasing and forcing and retracting said needle mounting and said needle into said barrel when said latch member of said latch device is disengaged from said needle mounting.

2. The syringe as claimed in claim 1, wherein said plunger includes a guide channel formed therein, and said follower includes a projection slidably engaged with said guide channel of said plunger and for guiding said follower to move relative to said plunger.

3. The syringe as claimed in claim 1, wherein said needle mounting includes a retaining member for releasably engaging with said latch member of said latch device which is capable or releasing said needle mounting from said barrel for allowing said needle mounting to be released and detached from said barrel and to be engaged into said chamber of said barrel when said latch member of said latch device is disengaged from said needle mounting.

4. The syringe as claimed in claim 1, wherein said latch device includes an inclined surface formed in said latch member for being actuated to disengage said latch member of said latch device from said needle mounting.

5. The syringe as claimed in claim 4, wherein said barrel includes a gasket disposed therein for selectively engaging with said plunger which selectively forces said gasket to engage with said inclined surface of said latch member.

6. The syringe as claimed in claim 1, wherein said barrel includes a space formed therein for receiving and engaging with said latch device.

7. The syringe as claimed in claim 1, wherein said latch device is resiliently secured to said barrel with a support member.

8. The syringe as claimed in claim 1, wherein said barrel includes an anchoring member provided in said mouth, and said needle includes an engaging member for releasably engaging with said anchoring member of said barrel.

9. The syringe as claimed in claim 1, wherein said piston includes at least one rib extended radially and outwardly therefrom for engaging with said plunger and for releasably attaching to said plunger.

10. The syringe as claimed in claim 1, wherein said piston includes an extension extended outwardly therefrom for selectively engaging with said catch device of said follower and for allowing said follower to be selectively anchored and secured to said piston when said plunger is pulled outwardly relative to said barrel.
